(19) United States
(12) Patent Application Publication
(10) Pub. No.: US 2023/0080297 A1
(43) Pub. Date: Mar. 16, 2023

(54) Cosmetic Material Composition Comprising Solid Phase Polymer Having Improved Solubility

(71) Applicant: LG Household & Health Care Ltd., Seoul (KR)

(72) Inventors: Koo Chul Kwon, Seoul (KR); Dong Hyun Kim, Seoul (KR); Nam Seo Son, Seoul (KR); Sang Wook Park, Seoul (KR)

(73) Assignee: LG Household & Health Care Ltd., Seoul (KR)

(21) Appl. No.: 17/639,699

(22) PCT Filed: Sep. 2, 2020
(86) PCT No.: PCT/KR2020/011785

§ 371 Date: Aug. 5, 2022

(30) Foreign Application Priority Data
Sep. 3, 2019 (KR) 10-2019-0109138
May 15, 2020 (KR) 10-2020-0058055

(51) Int. Cl. A61K 8/44; A61K 8/73; A61K 8/65; A61K 8/02; A61Q 5/00

(52) U.S. Cl. CPC A61K 8/44; A61K 8/735; A61K 8/65; A61K 8/731; A61K 8/736; A61K 8/732; A61K 8/0216; A61Q 5/002

(57) ABSTRACT

The present invention relates to a solid phase cosmetic composition comprising a freeze-dried component of a composition comprising a functional component and a basic amino acid. A solid phase component according to the present invention has excellent re-dissolvability, and exhibits insignificant changes in the hardness and pH of the composition for dissolving the solid component even after re-dissolution, and thus a cosmetic having excellent stability can be provided.

### TECHNICAL FIELD

The present invention relates to a cosmetic composition comprising a solid-phase, in which the solid phase comprises polymer components and has improved solubility.

### BACKGROUND ART

In cosmetics, polymers such as hyaluronic acid and collagen and functional ingredients such as vitamins and antioxidants have been generally used as active ingredients for skin improvement comprising whitening, wrinkle improvement, skin elasticity enhancement, moisturizing, skin regeneration promotion, removal of harmful oxygen, and the like. However, the active ingredients may increase the viscosity of cosmetics. In addition, when the active ingredients have a possibility of reacting with other ingredients or are present in an aqueous solution or in contact with water, they may be easily destroyed due to oxidation resulting from exposure to light or air, or the color or odor thereof may be changed. Therefore, the use of the active ingredients as cosmetics has been limited. As a method of solving the above problems, stabilizing active ingredients, and maximizing efficiency in skin improvement, formulation into a powder-type solid ingredient has been proposed (Patent Document 1). In the case of cosmetics providing the powder-type solid ingredient and the composition for dissolving the solid ingredient separately, in addition to the advantages described above, consumers may enjoy a new type of cosmetic where they get to mix the solid powder before use by themselves. However, in the case of cosmetics providing the powder-type solid ingredient (i) and composition for dissolving the solid ingredient (ii) separately, if (ii) is a liquid having low flowability such as a cream type composition, there may be difficulty in (i) penetrating into (ii) or hydration may be limited in the penetration process, leading to problems in re-dissolvability. Accordingly, there is a limitation in diversifying cosmetic formulations for the aforementioned type of cosmetics. Therefore, further research is required for the development of a solid-phase composition having improved solubility in various formulations in order to provide various cosmetic formulations.

### DISCLOSURE

### Technical Problem

In order to solve the above-described problem, to increase the re-dissolvability of water-dispersible polymer ingredients in an emulsion-type cosmetic composition with low flowability, the present invention provides a cosmetic composition, a cosmetic, or a cosmetic kit comprising a freeze-dried ingredient of a composition comprising a basic amino acid and a water-dispersible polymer.

### Technical Solution

One aspect of the present invention is to provide a solid-phase cosmetic composition comprising a freeze-dried ingredient, wherein the said freeze-dried ingredient comprises a water-dispersible polymer and a basic amino acid.

Since the freeze-dried ingredient of the present invention comprises a basic amino acid, when the freeze-dried ingredient is mixed with a composition for dissolving it, polymers comprised in the said composition for dissolving reacts with the said basic amino acid and separates water molecules, which in turn allows the functional ingredients comprised in the freeze-dried ingredient to smoothly re-dissolve into the said composition for dissolving. In the present invention, a solid phase means a solid state. A liquid-phase composition may be solidified through a process such as drying, crystallization, granulation, or the like and further processed through a process such as pulverization or the like so as to have a predetermined size. In the present invention, a freeze-dried ingredient is an example of the solid phase and encompasses both a freeze-dried product and a powder obtained by pulverizing the freeze-dried product.

The freeze-dried ingredient of the present invention may be prepared by dissolving a functional ingredient and a basic amino acid in a solvent to prepare an aqueous solution, solidifying the aqueous solution by cooling to form a solid phase composition, and decompressing the solid phase composition at a temperature below the triple point. When the solid phase composition is decompressed at a temperature below the triple point, water molecules comprised in the composition sublimes, meaning that they become a gaseous phase without going through a liquid phase. In this case, the functional ingredients and the basic amino acids maintain the water-dissolved structure and form a porous structure. The porous structure is a microstructure and may maintain its porosity without being destroyed during a pulverization process, which is a macroscopic change in properties.

The basic amino acid of the present invention encompasses both a basic amino acid and a derivative thereof. The basic amino acid comprises a functional group capable of bonding to the hydrophilic functional group comprised in a polymer ingredient, which is normally comprised in a liquid-phase formulation having high hardness, wherein the bonding forms water molecules to improve the re-dis solvability of the functional ingredient in the liquid-phase formulation having high hardness. As an example, the functional group of the basic acid may be an amine group (NH₃⁺), which may bond with a caboxyl group (—COO—) included in a polymer, which is normally comprised in a liquid-phase formulation such as a cream or the like. When the basic amino acid and an emulsion formulation are mixed, the functional group included in the basic amino acid may weaken the hydrogen bond between the hydrophilic functional group of the polymer in a liquid-phase formulation and the hydroxyl group (—OH) of water to elute water molecules and may allow the water molecules to be easily bonded to a solid ingredient to improve the hydration of the solid ingredient in the formulation, thereby improving the solubility or re-dissolvability of the solid ingredient.

In addition, since the basic amino acid has a structure including an amine group (NH₃^{±}) and also a carboxyl group or at least two hydroxyl groups, that is, a structure capable of serving as a proton donor and acceptor in one molecule, it may act as a buffer solution. For this reason, when the solid-phase cosmetic composition comprising the basic amino acid is mixed with a cosmetic composition having low flowability, the pH in the formulation is buffered despite an increase in the re-dissolvability of the solid phase, and accordingly, a decrease in flowability of the formulation may be prevented, and stability may be maintained.

The basic amino acid may be, for example, one or more selected from the group consisting of arginine, lysine, asparagine, histidine, citrulline, ornithine, tromethamine, and cysteamine, but the present invention is not limited thereto.

In the solid-phase cosmetic composition of the present invention, the basic amino acid may be comprised in an amount of 1 to 90% (w/w), specifically 5 to 35% (w/w), but the present invention is not limited thereto.

The functional ingredient is an ingredient comprised in a cosmetic material to achieve a predetermined purpose. In the present invention, the functional ingredient may be an ingredient having low re-dissolvability in a cosmetic composition in the form of an emulsion having low flowability.

Although there is no limitation in the number of the types of the functional ingredients, at least one type of a polymer ingredient may be comprised. Although there is no limitation related to the mass of the polymer, the polymer may have a mass of preferably 300 kDa or less, preferably 50 kDa or less. In the solid-phase cosmetic composition of the present invention, the functional ingredient may be comprised in an amount of 0.01% to 90% (w/w), preferably 10 to 80% (w/w).

The functional ingredient may be, for example, one or more of a water-dispersible polymer or a keratin care ingredient.

The water-dispersible polymer is a water-soluble polymer and may be, for example, one or more selected from the group consisting of collagen, hydrolyzed hyaluronic acid, hyaluronic acid, β-glucan, xanthan gum, carboxymethyl cellulose, a biosaccharide, cellulose, cellulose gum, arabic gum, gelatin, agar, carrageenan, algin, chitosan, starch, galactomannan, glucomannan, guar gum, locust bean gum, alginate, and gellan, but the present invention is not limited thereto.

The keratin care ingredient may be amino acids or hydroxy acids. The hydroxy acids may be α-hydroxy acids, β-hydroxy acids, or polyhydroxy acids. As a specific example, the keratin care ingredient may be one or more selected from the group consisting of serine, cysteine, threonine, glycine, tyrosine, aspartic acid, glutamic acid, histidine, carnitine, glycolate, lactate, malate, citrate, salicylate, and gluconolactate, but the present invention is not limited thereto.

In addition, the solid-phase cosmetic composition of the present invention may further comprise at least one type of a low-molecular-weight (1 kDa or less) functional ingredient. In the solid-phase cosmetic composition of the present invention, the low-molecular-weight functional ingredient may be comprised in an amount of 0.01 to 99% (w/w), specifically 30 to 80% (w/w), and more specifically 45 to 65% (w/w). Although there is no limitation related to the content of the low-molecular-weight functional ingredient, when the content increases, re-dissolvability may be improved, but it may not provide the effects of a high-molecular-weight functional ingredient, such as adhesion to skin, moisturizing, and elasticity. Therefore, the content of the low-molecular-weight functional ingredient needs to be determined according to a polymer content.

Although there is no limitation related to the pH of the solid-phase cosmetic composition of the present invention, it is possible for the pH thereof to be 4.0 to 9.0, and a pH of 6.0 to 8.0 is preferable. When the pH is above or below the above-described range, the stability of the active ingredient may be degraded, or skin irritation may be caused.

The solid-phase cosmetic composition of the present invention which comprises a functional ingredient and a basic amino acid may further comprise a hydrophilic amino acid and/or an acidic amino acid. When a hydrophilic or acidic amino acid is comprised, it may buffer the pH increase caused by the basic amino acid when the freeze-dried ingredient is mixed with the emulsion composition for dissolving the solid ingredient.

The hydrophilic amino acid encompasses a derivative of an amino acid and may be, for example, one or more selected from the group consisting of serine, threonine, tyrosine, cysteine, asparagine, and glutamine, but the present invention is not limited thereto.

The acidic amino acid is an amino acid whose side branch has COOH, encompasses a derivative of an amino acid, and may be, for example, one or more selected from the group consisting of aspartic acid and glutamic acid, but the present invention is not limited thereto.

Another aspect of the present invention is to provide a cosmetic composition comprising: a first composition in a solid phase comprising a composition which comprises a water-dispersible polymer and a basic amino acid, wherein the said composition comprising a water-dispersible polymer and a basic amino acid is freeze-dried; and a second composition in the form of an emulsion having a hardness of 5 dyne/cm² or more.

The cosmetic composition may be a two-agent type composition comprising the first composition and the second composition, respectively.

Still another aspect of the present invention is to provide a cosmetic comprising a first composition and a second composition.

The first composition is in a solid phase and comprises a freeze-dried ingredient of a composition comprising a functional ingredient and an amino acid, and the second composition is intended to dissolve the first composition and is in the form of an emulsion.

In the cosmetic of the present invention, the first composition and the second composition may be comprised in separate forms. The second composition intended to dissolve the first composition is in the form of an emulsion, and there is no limitation related to the type thereof. Preferably, the second composition of the present invention is in the form of an emulsion having high hardness and low flowability and may be a cream, gel, or lotion formulation. When the functional ingredient comprised in the first composition is, for example, a polymer, the re-dis solvability of the freeze-dried ingredient into an emulsion that does not contain sufficient moisture, such as a cream, is very low. The cosmetic material of the present invention solves this problem by comprising a basic amino acid in the first composition and freeze-drying it. In the present invention, the flowability of the emulsion formulation may be defined by hardness, and the emulsion formulation may have a hardness of 5 dyne/cm² or more, 5 to 100 dyne/cm², 10 to 50 dyne/cm², or 25 to 35 dyne/cm².

Moreover, the second composition in the form of an emulsion may not exhibit a decrease in hardness of 30% or more or exhibit a decrease in hardness of 15% or less when mixed with the first composition. When two formulations are mixed for use, differentiated efficacy and feeling of use may be provided to consumers without affecting the stability of the second composition over time.

Although there is no limitation to the pH of the cosmetic composition of the present invention, it is possible for the pH to be 4.0 to 9.0, and a pH of 6.0 to 8.0 is preferable. When the pH is above or below the above-described range, the stability of the active ingredient may be degraded, and skin irritation may be caused.

The cosmetic may be a two-agent type cosmetic comprising the first composition and the second composition, respectively.

Yet another aspect of the present invention provides a kit for preparing a cosmetic composition, which comprises: a first composition in a solid phase comprising a composition which comprises a water-dispersible polymer and a basic amino acid, wherein the said composition comprising a water-dispersible polymer and a basic amino acid is freeze-dried; and a second composition in the form of an emulsion having a hardness of 5 dyne/cm² or more.

Yet another aspect of the present invention provides a cosmetic material kit, which comprises: a first composition in a solid phase comprising a composition which comprises a water-dispersible polymer and a basic amino acid, wherein the said composition comprising a water-dispersible polymer and a basic amino acid is freeze-dried; and a second composition in the form of an emulsion having a hardness of 5 dyne/cm² or more.

In the kit of the present invention, the first composition and the second composition may be comprised in separate forms. That is, the cosmetic material kit may be a two-agent type cosmetic material kit comprising the first composition and the second composition, respectively.

In order to avoid overlapping description, the descriptions related to the cosmetic composition and cosmetic described above may also be applied to the kit for preparing a cosmetic material.

Yet another aspect of the present invention provides a method of preparing a two-agent type cosmetic, which comprises: preparing a first composition in a solid phase comprising a composition which comprises a functional ingredient and a basic amino acid, wherein the said composition comprising a functional ingredient and a basic amino acid is freeze-dried, comprising: dissolving a functional ingredient and a basic amino acid in a solvent to prepare an aqueous solution; freeze-drying the aqueous solution by cooling at −20° C. to −180° C.; and preparing a second composition in the form of an emulsion having a hardness of 5 dyne/cm² or more.

The cosmetic preparation method may be a method of preparing a two-agent type cosmetic, which comprises a freeze-dried solid ingredient having improved re-dissolvability. By comprising both the functional ingredient and the basic amino acid, the freeze-dried solid ingredient comprised in the first composition of the present invention may have excellent solubility in a dissolution formulation even when re-dissolved after the freeze-drying.

The freeze drying may be performed by solidifying an aqueous solution by cooling, in which the functional ingredient and the basic amino acid are dissolved, and decompressing the solidified aqueous solution at a temperature below the triple point. The freeze drying may be performed by cooling at −20° C. to −180° C.,−100° C. to −170° C., or −120° C. to −170° C. When the cooling is performed at a temperature within the above-described range, the quality of the freeze-dried product can be improved, and thus it is advantageous for use as a cosmetic material.

In addition, the freeze drying may further comprise pulverizing the freeze-dried solid ingredient. When the solid phase composition is decompressed at a temperature below the triple point, water molecules comprised in the composition sublimes, meaning that they enter a gaseous phase without going through a liquid phase. In this case, the functional ingredients and the basic amino acids maintain the water-dissolved structure and form a porous structure. The porous structure is a microstructure and may maintain its porosity without being destroyed during the pulverization process, which is a macroscopic change in properties. The solvent may be a solvent generally used to prepare a cosmetic material or a cosmetic. As a preferred example, the solvent may be water such as purified water.

The functional ingredient may be one or more selected from the group consisting of collagen, hyaluronic acid, hydrolyzed hyaluronic acid, β-glucan, xanthan gum, carboxymethyl cellulose, a biosaccharide, cellulose, cellulose gum, arabic gum, gelatin, agar, carrageenan, algin, chitosan, starch, galactomannan, glucomannan, guar gum, locust bean gum, alginate, and gellan.

The basic amino acid may be one or more selected from the group consisting of arginine, lysine, asparagine, citrulline, ornithine, tromethamine, and cysteamine.

In order to avoid overlapping description, the descriptions about the cosmetic material composition and cosmetic described above may also be applied to the method of preparing a cosmetic.

### Advantageous Effects

The present invention relates to a solid-phase cosmetic composition comprising a freeze-dried ingredient of a composition comprising a functional ingredient and a basic amino acid, wherein the solid ingredient of the present invention has excellent re-dissolvability and exhibits insignificant changes in the hardness and pH of the composition for dissolving the solid ingredient even after re-dissolution, and therefore, a cosmetic having excellent stability can be provided.

### DESCRIPTION OF DRAWINGS

FIG. 1 is a diagram illustrating the mechanism in which a solid-phase composition of the present invention is dissolved in a liquid-phase formulation.

FIG. 2 is a diagram illustrating the change over time when a solid-phase composition according to an embodiment of the present invention is mixed with a liquid-phase formulation.

### MODES OF THE INVENTION

Hereinafter, examples will be provided to facilitate understanding of the present invention. However, the following examples are not intended to limit the present invention.

### Experimental Example 1. Preparation of Solid-Phase Composition and Cream Formulation

Solid-phase cosmetic compositions comprising arginine as an amino acid, and hydrolyzed hyaluronic acid (0.5 to 10 kDa, SK Bioland Co., Ltd, Korea) or hyaluronic acid (10 kDa or more, SK Bioland Co., Ltd, Korea) as a functional polymer were prepared according to Table 1.

TABLE 1

| | Content (w/w %) | | | | | |
|---|---|---|---|---|---|---|
| | | | Comparative | | | Comparative |
| Ingredient | Example 1 | Example 2 | Example 1 | Example 3 | Example 4 | Example 2 |
| Hydrolyzed | 25% | 25% | 25% | | | |
| hyaluronic acid | | | | | | |
| Hyaluronic acid | | | | 25% | 25% | 25% |
| Serine | 65% | 65% | 75% | 65% | 65% | 75% |
| Arginine | 10% | — | — | 10% | — | — |
| Lysine | — | 10% | — | — | 10% | — |

Specifically, ingredients shown in Table 1 were dissolved in an excessive amount of water to prepare an aqueous solution, and the prepared aqueous solution was solidified by rapid cooling at −150° C. to form a solid phase, followed by a freeze-drying process. The freeze-dried solid was pulverized for convenient use to prepare a solid-phase composition in a powder form of the present invention. When the solid phase composition is decompressed at a temperature below the triple point, water molecules comprised in the composition sublimes, meaning that they become a gaseous phase without going through a liquid phase. In this case, the remaining ingredients shown in Table 1 maintain the water-dissolved structure and form a porous structure. The porous structure is a microstructure and may maintain its porosity without being destroyed during the pulverization process, which is a macroscopic change in properties. A second composition for dissolving the solid-phase cosmetic composition was prepared according to Table 2.

TABLE 2

| Ingredient | Content (w/w) |
|---|---|
| Stearyl alcohol | 4 |
| Hydrogenated polydecene | 8.5 |
| PEG-40 stearate | 2.5 |
| Dimethicone | 6.5 |
| Water | 60.9 |
| Glycerin | 17 |
| Acrylate/C10-30 | 0.2 |
| alkylacrylate crosspolymer | |
| Carbomer | 0.1 |
| Tromethamine | 0.3 |
| Total | 100 |

As a composition to dissolve the above prepared solid phase composition, a cosmetic composition as a cream having low flowability was prepared according to Table 2. The pH and hardness of this cream composition was 7.15 and 30 dyne/cm², where the hardness was measured using a hardness meter immediately after preparation. The hardness was measured using an indentation hardness test method which is generally widely used. The indentation hardness test method is a test method in which the cosmetic material is pressed using a presser with a constant test load for a certain period of time to obtain the degree of indentation in numerical terms, and a Fudoh rheometer (Rheo Tech Co., Ltd., Japan) was used as the hardness meter.

### Test Example 1. Confirmation of Change in Physical Properties of Composition

Each of the solid-phase cosmetic compositions prepared in Examples 1 and 2 and Comparative Example 1 was mixed with the cream composition having a hardness of 30 dyne/cm², which was prepared in Experimental Example 1, and the re-dissolvability and re-dissolution time of the solid-phase composition, and the changes in pH and hardness of the cream composition were examined. A portion of each solid-phase composition (Examples 1 and 2 and Comparative Example 1) were taken by a spatula or by hand and mixed with the cream composition by hand to confirm the re-dissolution. (FIG. 2).

TABLE 3

| | | | |
|---|---|---|---|
| | | Hardness | |
| | | (dyne/cm²) | |
| Re- | Re-dissolution | (variation relative | |
| dissolvability | time (s) | to initial cream) | PH |

| Example 1 | ⊚ | 15 s | 25 (83%) | 7.12 | |
|---|---|---|---|---|---|
| Example 2 | ⊚ | 17 s | 27 (90%) | 7.13 | |
| Comparative | ◯ | 35 s | 29 (97%) | 7.15 | |
| Example 1 | | | | | |
| Example 3 | Δ | 112 s | 30 (100%) | 7.14 | |
| Example 4 | Δ | 125 s | 31 (103%) | 7.13 | |
| Comparative | X | not | 31 (103%) | 7.16 | |
| Example 2 | | re-dissolved | | | |
| (⊚ very good, the powder did not agglomerate, and the surface of the composition after re-dissolution was very uniform, ◯: good, trace amounts of powder agglomeration were seen, and the surface of the composition after re-dissolution was uniform, | | | | | : slightly poor, the surface of the composition after re-dissolution was slightly non- uniform and agglomeration was visible, X: very poor, re-dissolution was not reached, and agglomeration was severe) |

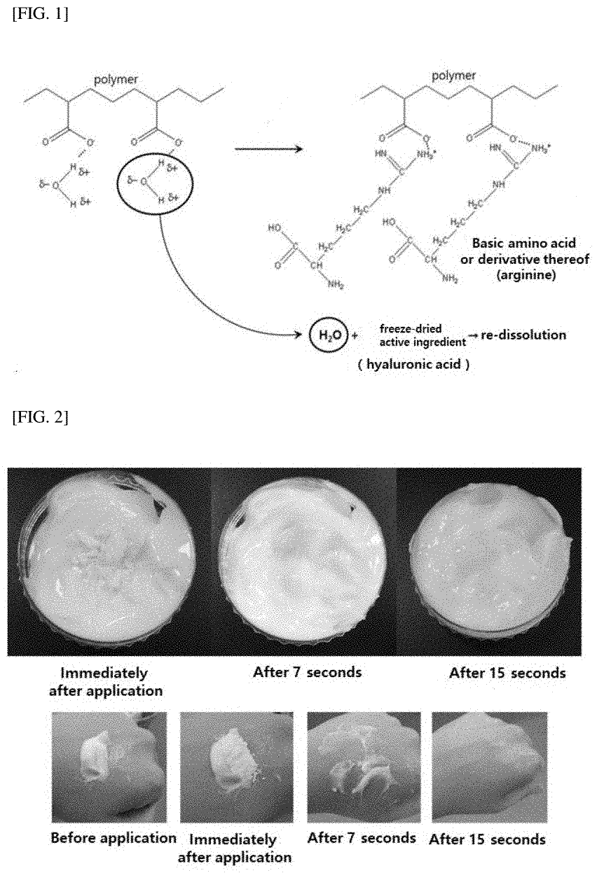

As shown in Table 3, in the case of the solid-phase compositions of Examples 1 and 2 which are compositions comprising ultra-low-molecular-weight hydrolyzed hyaluronic acid and a basic amino acid, it can be confirmed that the solid ingredient was re-dissolved in the cream having low flowability at a high rate via the mechanism shown in FIG. 1. However, it was confirmed that in the case of the solid ingredient of Comparative Example 1 which was formed only of a functional polymer without the use of a basic amino acid, the re-dissolution time was at least twice as long compared to Examples 1 and 2, wherein the re-dissolution time was determined based on measuring how long it took for the solid ingredient to be invisible and unfelt, thus being a uniformly mixed state. In the case of the compositions comprising high-molecular-weight hyaluronic acid, the solid-phase ingredient agglomerated severely and did not re-dissolve as in Comparative Example 2. It can be confirmed that when a basic amino acid was used as in Examples 3 and 4, the improvement in re-dissolvability of the present invention could be reached, but hydrolyzed hyaluronic acid is more preferred to have the excellent quality of a cosmetic.

### Experimental Example 2. Preparation of Solid-Phase Composition Comprising Various Functional Polymer Ingredients

In order to confirm the improve in re-dissolvability of the present invention based on the use of various functional polymer ingredients other than hydrolyzed hyaluronic acid as provided in Examples 1 and 2, solid-phase cosmetic compositions were prepared according to Table 4.

TABLE 4

| | Content (w/w %) | | | | | |
|---|---|---|---|---|---|---|
| | | | | Comparative | Comparative | Comparative |
| Ingredient | Example 5 | Example 6 | Example 7 | Example 3 | Example 4 | Example 5 |
| β-glucan | 30% | — | — | 30% | — | — |
| Xanthan gum | — | 30% | — | — | 30% | — |
| Carboxymethyl | — | — | 30% | — | — | 30% |
| cellulose | | | | | | |
| Serine | 60% | 60% | 60% | 70% | 70% | 70% |
| Arginine | 10% | 10% | 10% | — | — | — |

### Test Example 2. Confirmation of Re-Dissolvability of Solid-Phase Composition Comprising Various Functional Polymer Ingredients

In order to confirm the re-dissolvability of the solid-phase compositions of Examples 5 to 7 and Comparative Examples 3 to 5 which were prepared in Experimental Example 2, they were mixed with the cream composition having a hardness of 30 dyne/cm², which was prepared according to Table 2 in Experimental Example 1, and the re-dissolvability, the re-dissolution rate improvement (%), and the changes in pH and hardness were observed. Specifically, the rate of improvement in the re-dissolution rate was confirmed by comparing the re-dissolution rate (1/re-dissolution time) of the experimental groups that comprise basic amino acid (Examples 5 to 7) with the experimental groups which do not comprise basic amino acid (Comparative Example 3 or 5), where all experimental groups included the same polymer. In addition, the variation in hardness was confirmed by measuring the hardness immediately after mixing the solid-phase composition and the cream composition and comparing it to the initial hardness (30 dyne/cm²) of the cream composition.

TABLE 5

| | | | Hardness | | |
|---|---|---|---|---|---|
| | | | (dyne/cm²) | | |
| | | | (variation relative | | |
| | | Re-dissolution | to initial cream | | |
| | Re-dissolvability | time (s) | hardness) | PH | |
| Example 5 | ⊚ | 18 s | 24 (80%) | 7.11 | |
| Example 6 | ⊚ | 38 s | 27 (90%) | 7.14 | |
| Example 7 | ⊚ | 32 s | 26 (87%) | 7.15 | |
| Comparative | Δ | 58 s | 30 (100%) | 7.15 | |
| Example 3 | | | | | |
| Comparative | Δ | 64 s | 30 (100%) | 7.14 | |
| Example 4 | | | | | |
| Comparative | Δ | 58 s | 30 (100%) | 7.15 | |
| Example 5 | | | | | |
| (⊚ very good, the powder did not agglomerate, and the surface of the composition after re-dissolution was very uniform, ◯: good, trace amounts of powder agglomeration were seen, and the surface of the composition after re-dissolution was uniform, | | | | | : slightly poor, the surface of the composition after re-dissolution was slightly non-uniform and agglomeration was visible, X: very poor, re-dissolution was not reached, and agglomeration was severe) |

TABLE 6

| | Rate of improvement in |
|---|---|
| | re-dissolution rate (%) |
| | (relative to re-dissolution rate |
| | of Comparative Examples 1, |
| | 3, 4, and 5 which do |
| Type of polymer | not comprise basic amino acid) |
| Hydrolyzed hyaluronic acid | 233.3% |
| β-glucan | 322.2% |
| Xanthan gum | 168.4% |
| Carboxymethyl cellulose | 181.3% |

### Test Example 3. Confirmation of Re-Dissolvability in Various Preparations

In order to confirm that the re-dissolvability of the solid-phase composition of the present invention is improved in various preparations other than the cream composition according to Table 2 (hardness: 30 dyne/cm²), the re-dissolvability of the solid-phase composition of Example 1 was compared with Comparative Example 1 (which does not include basic amino acid) when mixed with an essence (8100 cps) or a solubilized toner (3800 cps). The essence and toner were prepared according to the compositions shown in Table 7 and Table 8, respectively, and evaluated. The viscosity of the essence was measured at 30 rpm for 60 seconds using a Brookfield viscometer (Brookfield LV, Brookfield Engineering Laboratories, Inc., MA, USA). To reduce the effect of air bubbles, the viscosity was measured after defoaming.

TABLE 7

| Ingredient | Content (w/w) |
|---|---|
| PEG/PPG/polybutylene glycol-8/5/3glycerin | 2 |
| Sodium polyacrylate | 0.2 |
| Carbomer | 0.18 |
| Acrylate/C10-30alkylacrylate crosspolymer | 0.05 |
| PEG-150 | 1.2 |
| Glycerin | 11 |
| Cyclopentasiloxane | 1 |
| Dimethicone | 6.26 |
| PEG-60 hydrogenated castor oil | 0.4 |
| Tromethamine | 0.21 |
| 1,2-Hexanediol | 1.5 |
| Dipropylene glycol | 8 |
| Water | 68 |
| Total | 100 |

TABLE 8

| Ingredient | Content (w/w) |
|---|---|
| Water | 78 |
| Alcohol | 1 |
| Glycerin | 9.5 |
| PEG-40 hydrogenated castor oil | 0.6 |
| Carbomer | 0.08 |
| Dipropylene glycol | 6.64 |
| 1,2-Hexanediol | 2 |
| Dimethicone | 2 |
| Ammonium acryloyldimethyltaurate/ | 0.1 |
| VP copolymer | |
| Tromethamine | 0.08 |
| Total | 100 |

TABLE 9

| | | Improvement of |
|---|---|---|
| | Re-dissolvability | re-dissolution rate (%) |
| Cream | ⊚ | 234.5% |
| Essence | ⊚ | 122.7% |
| Toner (solubilized) | ⊚ | 105.8% |

As shown in Table 9, in the case of the solid-phase composition in which both a functional polymer and a basic amino acid were freeze-dried, it can be confirmed that the re-dissolution rates were improved in all of the cream, essence, and toner formulations as compared to when the solid-phase composition of Comparative Example 1 was mixed with each formulation, and particularly, the degree of improvement of the re-dissolution rate in the cream formulation was higher than that in the solubilized toner where the re-dissolution of the freeze-dried polymer was not problematic due to the low viscosity/hardness of the formulation itself.

### Test Example 4. Confirmation of Re-Dissolution Improvement Effect According to Type of Amino Acid

In order to confirm whether the re-dissolution improvement effect of the freeze-dried solid composition is obtained even with the use of neutral and acidic amino acids other than the basic amino acid of the present invention, glutamic acid as an acidic amino acid, serine as a neutral amino acid, and arginine as a basic amino acid were freeze-dried with a functional polymer as shown in Table 10, and the change in re-dissolvability and hardness in the cream preparation according to Table 2 was confirmed.

TABLE 10

| | Content (w/w%) | | |
|---|---|---|---|
| Ingredient | Example 6 | Example 7 | Example 8 |
| Hydrolyzed hyaluronic acid | 70% | 70% | 70% |
| Glutamic acid | 30% | — | — |
| Serine | — | 30% | — |
| Arginine | — | — | 30% |

TABLE 11

| | | Re-dissolution | Hardness | | |
|---|---|---|---|---|---|
| | Re-dissolvability | time (s) | (dyne/cm²) | PH | |
| Example 6 | ◯ | 20 s | 27 | 6.89 | |
| Example 7 | Δ | 25 s | 30 | 7.15 | |
| Example 8 | ⊚ | 12 s | 26 | 7.45 | |
| (⊚ very good, the powder did not agglomerate, and the surface of the composition after re-dissolution was very uniform, ◯: good, trace amounts of powder agglomeration were seen, and the surface of the composition after re-dissolution was uniform, | | | | | : slightly poor, the surface of the composition after re-dissolution was slightly non-uniform and agglomeration was visible, X: very poor, re-dissolution was not reached, and agglomeration was severe) |

As shown in Table 10, it was confirmed that the neutral amino acid did not exhibit any improvement in re-dissolvability, and that the acidic amino acid and the basic amino acid improved re-dissolvability and re-dissolution time. However, in the case of the acidic amino acid, it was confirmed that the improvement in re-dissolvability of the polymer (carbomer) ingredient was caused by the presence of acidic amino acid reducing the hardness of the composition, and it can be seen that the acidic amino acid is less effective than basic amino acid in terms of weakening the binding force between the polymer (in the solid phase) and water.

1. A solid-phase cosmetic composition comprising a freeze-dried ingredient, wherein the freeze-dried ingredient comprises a functional ingredient and a basic amino acid.

2. The solid-phase cosmetic composition of claim 1, wherein the functional ingredient is one or more of a water-dispersible polymer or a keratin care ingredient.

3. The solid-phase cosmetic composition of claim 2, wherein the water-dispersible polymer is one or more selected from the group consisting of collagen, hyaluronic acid, hydrolyzed hyaluronic acid, β-glucan, xanthan gum, carboxymethyl cellulose, a biosaccharide, cellulose, cellulose gum, arabic gum, gelatin, agar, carrageenan, algin, chitosan, starch, galactomannan, glucomannan, guar gum, locust bean gum, alginate, and gellan.

4. The solid-phase cosmetic composition of claim 1, wherein the basic amino acid is one or more selected from the group consisting of arginine, lysine, asparagine, citrulline, ornithine, tromethamine, and cysteamine.

5. The solid-phase cosmetic composition of claim 1, wherein the composition further comprises a hydrophilic amino acid and/or an acidic amino acid.

6-10. (canceled)

11. A cosmetic comprising:
a first composition in a solid phase, and the first composition comprises a freeze-dried ingredient comprising a functional ingredient and a basic amino acid; and
a second composition in the form of an emulsion, and the second composition has a hardness of 5 dyne/cm² or more.

12-15. (canceled)

16. A method for improving the re-dissolvability of a functional ingredient, comprising:
a step of preparing an aqueous solution comprising a functional ingredient and a basic amino acid; and
a step of freeze drying the aqueous solution into a solid-phase cosmetic composition comprising the freeze-dried ingredient.

17. The method of claim 16, further comprising a step of mixing the solid-phase cosmetic composition and an emulsion composition having a hardness of 5 dyne/cm² or more.
